(19) United States
(12) Patent Application Publication
(10) Pub. No.: US 2024/0369942 A1
(43) Pub. Date: Nov. 7, 2024

(54) Method for Optimizing Negative Tone Development Photoresist Model

(71) Applicant: Dongfang Jingyuan Electron CO., LTD., Beijing (CN)

(72) Inventor: Shijia GAO, Beijing (CN)

(73) Assignee: Dongfang Jingyuan Electron CO., LTD., Beijing (CN)

(21) Appl. No.: 18/772,311

(22) Filed: Jul. 15, 2024

Related U.S. Application Data
(63) Continuation-in-part of application No. PCT/CN2022/115976, filed on Aug. 30, 2022.

(30) Foreign Application Priority Data
Jan. 13, 2022 (CN) 202210040092.0

(51) Int. Cl. G03F 7/00; G03F 7/038; G03F 7/30; G06F 30/17

(52) U.S. Cl. CPC G03F 7/705; G03F 7/30; G06F 30/17; G03F 7/038

(57) ABSTRACT

A method for optimizing a negative tone development photoresist model, which includes: creating a concentration distribution function of acid in the photoresist based on light field distribution in the initial negative tone development photoresist model and acid concentration in photoresist; creating a concentration distribution function of a developing solution based on the concentration distribution function of the acid in the photoresist; creating a formula for calculating concentration diffusion of the developing solution based on the concentration distribution function of the developing solution to calculate diffusion results of developing solutions in different concentrations; simulating a development process through the formula for calculating concentration diffusion of the developing solution to obtain a pattern of simulated negative tone development photoresist after development; and comparing relevant data of the pattern of simulated negative tone development photoresist with that of a preset pattern.

### CROSS-REFERENCE TO RELATED APPLICATIONS

This application is a continuation-in-part of International (PCT) Patent Application No. PCT/CN2022/115976, filed on Aug. 30, 2022, which claims priority of China Patent Application No. 202210040092.0, filed on Jan. 13, 2022. The contents of the above-identified applications are incorporated herein by reference.

### TECHNICAL FIELD

The present disclosure is related to semiconductor technologies, and especially related to a method for optimizing a negative tone development photoresist model.

### BACKGROUND OF THE INVENTION

A lithography process is the most important manufacturing process in manufacturing process of modern very large scale integrated circuit (VLSI) circuits, and is an important means of transferring design patterns of integrated circuits on masks to the silicon wafer through a lithography machine. In the manufacturing process, as feature sizes gradually decrease, process windows available for manufacturing will become smaller and smaller. However, because the whole lithography process needs to be precisely controlled, requirements for accuracy of calculating of lithography are getting higher and higher. Accurate calculation of the lithography model can theoretically explore ways to increase lithography resolution and process window, and guide the optimization of process parameters.

In currently available technical solutions, an advanced photoresist technology is use of negative development. The photoresist used in negative development has good adhesion ability and blocking effect, and the light sensitivity speed is fast. However, it will be deformed and expanded during development. Compared with positive development, the thermal shrinkage effect of negative development is stronger, and the thermal shrinkage effect will make the distribution of the developing solution different from the light field distribution during the development process, resulting in a decrease in modeling accuracy. In addition, for chips, a size of a chip can reach up to 32 mm×26 mm, the line width of the smallest pattern may only be 10 nm, and a layout file of a lithography layer can reach hundreds of GB, so the model speed is a very key technical indicator. However, the existing optimization methods of negative tone development photoresist models are difficult to take into account both accuracy and speed.

### SUMMARY OF THE INVENTION

To overcome the technical problem that the existing optimization method of negative tone development photoresist models is difficult to take into both account accuracy and speed, the present disclosure provides a method for optimizing a negative tone development photoresist model.

The technical solution of the present disclosure to solve the above-mentioned technological problems is to provide a method for optimizing a negative tone development photoresist model, which includes following steps:
obtaining an initial negative tone development photoresist model;
creating a concentration distribution function S of acid in the photoresist based on light field distribution in the initial negative tone development photoresist model and acid concentration in photoresist;
creating a concentration distribution function D of a developing solution based on the concentration distribution function S of the acid in the photoresist; wherein concentration consumption of the developing solution is proportional to concentration consumption of the acid in the photoresist;
creating a formula R for calculating concentration diffusion of the developing solution based on the concentration distribution function D of the developing solution to calculate diffusion results of developing solutions in different concentrations;
simulating a development process through the formula R for calculating concentration diffusion of the developing solution to obtain a pattern of simulated negative tone development photoresist after development; and
comparing relevant data of the pattern of simulated negative tone development photoresist with that of a preset pattern, and taking the pattern of simulated negative tone development photoresist as a formal pattern of negative tone development photoresist if preset criteria are met.

Optionally, said creating the concentration distribution function S of the acid in the photoresist based on the light field distribution in the initial negative tone development photoresist model and the acid concentration in photoresist includes following steps:
obtaining data on the light field distribution based on positions of pixel points of a mask pattern from the initial negative tone development photoresist model, and constructing a light field distribution function E (x, y) based on information on the positions of the pixel points according to obtained data on the light field distribution, where E is a function related to (x, y) and (x, y) represents a position of a pixel point; and
creating a concentration distribution function S(x, y) of the acid in the photoresist, the concentration distribution function S(x, y) of the acid in the photoresist is relevant to the light field distribution function E(x, y) and S(x, y)=F(E(x, y)).

Optionally, the formula of the concentration distribution function S of the acid in the photoresist is S=1−G, where G is concentration of a photoacid-producing agent, an expression for instantaneous consumption rate of the concentration of the photoacid-producing agent is

$$\frac{\partial G}{\partial t} = - cGE ,$$

where c represents an exposure rate constant and t represents time.

Optionally, said creating the concentration distribution function D of the developing solution based on the concentration distribution function S of the acid in the photoresist includes following steps:
determining a consumption ratio of the acid in the photoresist and the developing solution according to a chemical reaction formula of the acid in the photoresist and the developing solution; and
creating the concentration distribution function D(x, y)=F(S(x, y)) according to the consumption ratio of the acid in the photoresist and the developing solution.

Optionally, an expression of the concentration distribution function of the developing solution is

$$\frac{\partial D}{\partial t} = - {K}_{amp} DS ,$$

where Kₐₘₚ represents a crosslinking rate constant and t represents time.

Optionally, before said creating the formula R for calculating concentration diffusion of the developing solution based on the concentration distribution function D of the developing solution to calculate diffusion results of developing solutions in different concentrations, the method further includes following steps:
creating an expression D1 representing a concentration diffusion direction of the developing solution; and
creating an expression D2 representing a concentration diffusion intensity of the developing solution.

Optionally, the expression representing a concentration diffusion direction of the developing solution is D1=D(x₁, y₁)*D(x₂, y₂), the expression representing a concentration diffusion intensity of the developing solution is D2=D(x₁, y₁)*(x₁-x₂, y₁−y₂).

Optionally, said creating the formula R for calculating concentration diffusion of the developing solution based on the concentration distribution function D of the developing solution to calculate diffusion results of developing solutions in different concentrations comprises following steps:
obtaining the diffusion direction and the diffusion intensity of the developing solution in each unit area according to the expression D1 representing a concentration diffusion direction of the developing solution and the expression D2 representing a concentration diffusion intensity of the developing solution; calculating diffusion results of the developing solution in each unit area based on the concentration distribution function D of the developing solution; constructing the calculation formula R(x, y) of the concentration diffusion of the developing solution based on the expression D1 representing a concentration diffusion direction of the developing solution and the expression D2 representing a concentration diffusion intensity of the developing solution; and adding up the diffusion results of the developing solution in each unit area to simulate diffusion of the developing solution.

Optionally, the formula for calculating a total diffusion result is R(x, y)=Σᵢ₌ₘ^{D(x}^{ᵢ}^{, y}^{ᵢ}^{)*D(x}^{₁}^{, y}^{₁}^{>0}D(xᵢ, yᵢ)*(x₁−xᵢ, y₁−yᵢ), where m is a positive integer constant; and the formula R(x, y)=Σᵢ₌ₘ^{D(x}^{ᵢ}^{, y}^{ᵢ}^{)*D(x}^{₁}^{, y}^{₁}^{)<0}D(xᵢ−yᵢ)*(x₁−xᵢ, y₁−yᵢ) is created based on an idea of calculus.

Optionally, comparison factors for comparing the pattern of simulated negative tone development photoresist after development with that of the preset pattern comprises root mean square and/or grid point error of a preset key dimension.

Compared with the prior art, the method for optimizing the negative tone development photoresist model of the present disclosure has following advantages:
1. The method for optimizing the negative tone development photoresist model of the present disclosure is a method for simulating the negative tone developing based on concentration distribution of the developing solution. Firstly, the concentration distribution function S of the acid in the photoresist is created based on relevant data on the light field distribution in the initial negative tone development photoresist model; Then, in the process of development, the developing solution will chemically react with the acid in the photoresist, and the developing solution will consume the acid in a corresponding proportion according to the chemical reaction formula. Therefore, it can be considered that the concentration consumption of the developing solution is proportional to the concentration consumption of the acid in the photoresist, based on which, the concentration distribution function D of the developing solution can be quickly created; Concentration distribution data of the developing solution can be obtained based on the concentration distribution function D of the developing solution. The developing solution distributed in different regions have different concentrations, which will produce diffusion effects. Therefore, the formula R for calculating concentration diffusion of the developing solution can be created based on the concentration distribution function D of the developing solution. Since in the development process, the photoresist chemically reacts with the developing solution, the change of the concentration of the developing solution is directly related to the image of the photoresist. In the negative tone development process, the part of the photoresist reacting with the developing solution will be retained, therefore, it can be known that which regions of the photoresist undergoes the reaction and the image of the photoresist after the reaction is thus known. Therefore, we can accurately simulate the change of photoresist in the development process by accurately simulating the change of the concentration distribution of the developing solution during the development process, so as to obtain a pattern with a high-precision of the negative tone development photoresist after development. This method can take into account both modeling speed and accuracy, and can make the modeling speed of the negative tone development comparable to that of forward development while ensuring accuracy.
2. The light field distribution function E in the present disclosure is constructed according to the obtained data on the light field distribution, and the concentration distribution function S of the acid in the photoresist is constructed based on the light field distribution function E, so when the light field distribution function E is a function related to (x, y), the concentration distribution function S of the acid in the photoresist is also a function related to (x, y), and this design is convenient for unifying variables, making it convenient for operation, and is conducive to reducing the amount of operation during optimization, and to improve the operation speed; Moreover, the data on the light field distribution in the present disclosure is obtained on the basis of positions of the pixel points of the mask pattern, which has a high accuracy.
3. The acid in the photoresist in the present disclosure is produced by decomposition of the photoacid-producing agent, so the expression of the distribution function of the acid concentration in the photoresist is S=1−G. Since the photoacid-producing agent will be decomposed under the light to produce the acid, instantaneous consumption rate of the concentration of the photoacid-producing agent is relevant to the exposure rate constant and the light field distribution function E.
4. The concentration distribution function D of the developing solution in the present disclosure is created according to the consumption ratio of the developing solution and the acid in the photoresist, and is a function related to (x, y), which is convenient for unifying variables, making it convenient for calculation, and is conducive to reducing the amount of operation during optimization, and improving the operation speed.
5. As mentioned in the first point above, in the process of development, the developing solution will chemically react with the acid in the photoresist, and the developing solution will consume the acid in a corresponding proportion according to the chemical reaction formula. Therefore, it can be considered that the concentration consumption of the developing solution is proportional to the concentration consumption of the acid in the photoresist. Therefore, the concentration distribution function of the developing solution is created using the instantaneous consumption rate ∂D/∂t of the developing solution, which is highly correlated with the instantaneous consumption rate ∂G/∂t of the photoacid-producing agent. Then, the concentration distribution function D of the developing solution is correlated with the concentration distribution function S of the acid in the photoresist through the crosslinking reaction rate constant Kₐₘₚ.
6. In the present disclosure, the developing solution in different positions has different concentrations, which may cause strong diffusion effect. The developing solution with a higher concentration may diffuse to the area where the developing solution with a lower concentration. When the concentration is greater than a certain intensity, the diffusion effect will be strengthened. The technical solution of the present disclosure can effectively and quickly calculate the sum of reactions by first creating the expression D1 of the concentration diffusion direction of the developing solution and the expression D2 of the concentration diffusion intensity of the developing solution, and then creating the calculation formula R of the concentration diffusion of the developing solution based on the idea of calculus, which greatly improves speed and accuracy of modeling.
7. In the present disclosure, both the expression D1 representing a concentration diffusion direction of the developing solution and the expression D2 representing a concentration diffusion intensity of the developing solution are expressions related to (x, y). The variables are unified, which is convenient for operation, and can further reduce the amount of operation during modeling, and improve the operation speed.
8. In the present disclosure, the formula R for calculating concentration diffusion of the developing solution is based on the idea of calculus to accumulate the diffusion of concentration, and does not need to be calculated by pure brute force superposition, which further reduces the amount of calculation during modeling and improves the overall calculation speed.
9. In the present disclosure, the developing state can be monitored by the expression D1=D(x₁, y₁)*D(x₂, y₂) of the concentration diffusion direction of the developing solution, and when the expression of the concentration diffusion direction of the developing solution becomes a fixed value, it indicates that the concentration distribution of the developing solution has entered a stable state and no longer diffuses.
10. The comparison factor in the present disclosure includes the root mean square and/or the grid point error of the preset key dimension, and the comparison is comprehensive, and the comparison error can be effectively avoided.

### BRIEF DESCRIPTION OF DRAWINGS

In order to more clearly illustrate the technical solutions in the embodiments of the present disclosure, the drawings that need to be used in descriptions of the embodiments or prior arts will be briefly discussed below, and it is obvious that the drawings in the following descriptions are only some embodiments of the present disclosure, and for those skilled in the art, other drawings can also be obtained according to these drawings without paying creative labor.

FIG. 1 is a block diagram of the method for optimizing a negative tone development photoresist model provided by the first embodiment of the present disclosure.

FIG. 2 is a light field distribution diagram of a selected photoresist region in the method for optimizing the negative tone development photoresist model provided by the first embodiment of the present disclosure.

FIG. 3 is a light field distribution diagram after diffusion of a developing solution in the selected photoresist region in the method for optimizing the negative tone development photoresist model provided by the first embodiment of the present disclosure.

FIG. 4 is a light field distribution diagram of another selected photoresist region in the method for optimizing the negative tone development photoresist model provided by the first embodiment of the present disclosure.

FIG. 5 is a light field distribution diagram after diffusion of a developing solution in said another selected photoresist region in the method for optimizing the negative tone development photoresist model provided by the first embodiment of the present disclosure.

### DETAILED DESCRIPTION OF THE INVENTION

In order to make the objects, technical solutions and advantages of the present disclosure clearer, the present disclosure will be further described in detail in combination with the drawings and the embodiments. It should be understood that the embodiments described herein are only used to explain the present disclosure and are not used to limit the present disclosure.

The terms “vertical”, “horizontal”, “left”, “right”, “up”, “down”, “top left”, “top right”, “lower left”, “lower right” and similar expressions are used herein for illustrative purposes only.

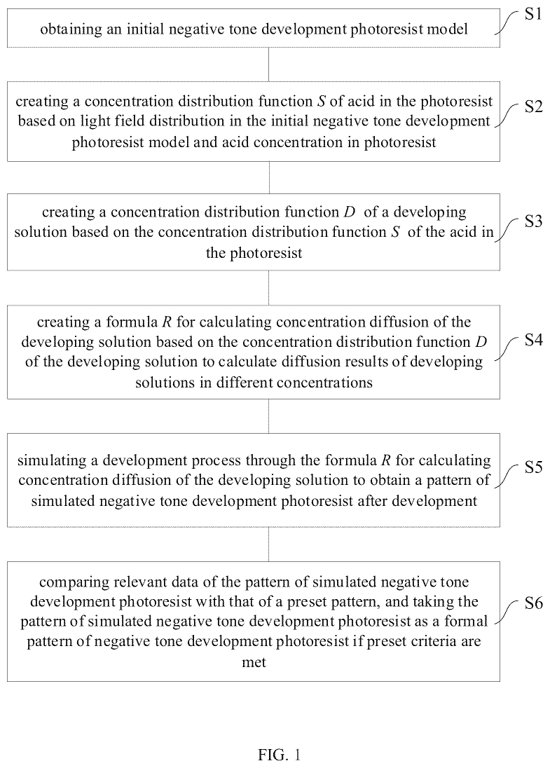

Please referring to FIGS. 1-5, a first embodiment of the present disclosure provides a method for optimizing the negative tone development photoresist model, the method includes following steps:
Step S1: obtaining an initial negative tone development photoresist model;
Step S2: creating a concentration distribution function S of acid in the photoresist based on light field distribution in the initial negative tone development photoresist model and acid concentration in photoresist;
Step S3: creating a concentration distribution function D of a developing solution based on the concentration distribution function S of the acid in the photoresist;
Step S4: creating a formula R for calculating concentration diffusion of the developing solution based on the concentration distribution function D of the developing solution to calculate diffusion results of developing solutions in different concentrations;
Step S5: simulating a development process through the formula R for calculating concentration diffusion of the developing solution to obtain a pattern of simulated negative tone development photoresist after development.
Step S6: comparing relevant data of the pattern of simulated negative tone development photoresist with that of a preset pattern, and taking the pattern of simulated negative tone development photoresist as a formal pattern of negative tone development photoresist if preset criteria are met.

It can be understood that the method for optimizing the negative tone development photoresist model of the present disclosure is a method for simulating the negative tone developing based on concentration distribution of the developing solution. Firstly, the concentration distribution function S of the acid in the photoresist is created based on relevant data on the light field distribution in the initial negative tone development photoresist model; Then, in the process of development, the developing solution will chemically react with the acid in the photoresist, and the developing solution will consume the acid in a corresponding proportion according to the chemical reaction formula. Therefore, it can be considered that the concentration consumption of the developing solution is proportional to the concentration consumption of the acid in the photoresist, based on which, the concentration distribution function D of the developing solution can be quickly created; Concentration distribution data of the developing solution can be obtained based on the concentration distribution function D of the developing solution. The developing solution distributed in different regions have different concentrations, which will produce diffusion effects. Therefore, the formula R for calculating concentration diffusion of the developing solution can be created based on the concentration distribution function D of the developing solution. Since in the development process, the photoresist chemically reacts with the developing solution, the change of the concentration of the developing solution is directly related to the image of the photoresist. In the negative tone development process, the part of the photoresist reacting with the developing solution will be retained, therefore, it can be known that which regions of the photoresist undergoes the reaction and the image of the photoresist after the reaction is thus known. Therefore, we can accurately simulate the change of photoresist in the development process by accurately simulating the change of the concentration distribution of the developing solution during the development process, so as to obtain a pattern with a high-precision of the negative tone development photoresist after development. This method can take into account both modeling speed and accuracy, and can make the modeling speed of the negative tone development comparable to that of forward development while ensuring accuracy.

Furthermore, step S1 specifically comprises the following steps:

Step S11: selecting at least a portion of the photoresist as a target area to generate a mask pattern in a predetermined size;

Specifically, in the embodiment, the mask pattern in the predetermined size is an image with a pixel size of 512*512. It should be understood that, the pixel size of the mask pattern can be adjusted according to actual needs.

Step S12: Creating an initial negative tone development photoresist model based on generated mask pattern in the predetermined size with existing modeling methods.

Additionally, step S2 specifically comprises the following steps:

Step S21: obtaining data on the light field distribution based on positions of pixel points of the mask pattern from the initial negative tone development photoresist model, and constructing a light field distribution function E(x, y) based on information on the positions of the pixel points according to obtained data on the light field distribution, where E is a function related to (x, y) and (x, y) is a position of a pixel point;

Specifically, the light field distribution function is a statistical distribution function of different light intensities caused by transmission, reflection or refraction at different positions. It is understandable that different optical models will have different light field distributions, and therefore, the light field distribution function E(x, y) created according to different optical models will also be different.

Step S22: creating a concentration distribution function S(x, y) of the acid in the photoresist, the concentration distribution function S(x, y) of the acid in the photoresist is relevant to the light field distribution function E(x, y) and S(x, y)=F(E(x, y)).

It is understandable that the light field distribution function E in the present disclosure is constructed according to the obtained data on the light field distribution, and the concentration distribution function S of the acid in the photoresist is constructed based on the light field distribution function E, so when the light field distribution function E is a function related to (x, y), the concentration distribution function S of the acid in the photoresist is also a function related to (x, y), and this design is convenient for unifying variables, making it convenient for operation, and is conducive to reducing the amount of operation during optimization, and to improve the operation speed; Moreover, the data on the light field distribution in the present disclosure is obtained on the basis of positions of the pixel points of the mask pattern, which has a high accuracy.

Furthermore, the formula of the concentration distribution function S of the acid in the photoresist is S=1−G, where G is concentration of a photoacid-producing agent, an expression for instantaneous consumption rate of the concentration of the photoacid-producing agent is

$$\frac{\partial G}{\partial t} = - cGE ,$$

where c represents an exposure rate constant and t represents time.

It can be understood that in mathematics, ∂G/∂t refers to the derivative of G with respect to t, where G is the concentration of the photoacid-producing agent and t represents time. Therefore, according to the definition of differentiation, ∂G/∂t refers to the instantaneous consumption rate of the concentration of the photoacid-producing agent.

It can be understood that the acid in the photoresist in the present disclosure is produced by decomposition of the photoacid-producing agent, so the expression of the distribution function of the acid concentration in the photoresist is S=1−G. Since the photoacid-producing agent will be decomposed under the light to produce the acid, instantaneous consumption rate of the concentration of the photoacid-producing agent is relevant to the exposure rate constant and the light field distribution function E.

The photoacid-producing agent is a kind of compounds that can be decomposed to generate specific acids under radiation such as light, gamma rays, and plasma. The generated acids can cause acid-sensitive resin to undergo decomposition or crosslinking reactions.

Furthermore, step S3 specifically comprises the following steps:

Step S31: determining a consumption ratio of the acid in the photoresist and the developing solution according to the chemical reaction formula of the acid in the photoresist and the developing solution;

It can be understood that in the process of development, the developing solution will chemically react with the acid in the photoresist, and the developing solution will consume the acid in a corresponding proportion according to the chemical reaction formula. Therefore, it can be considered that the concentration consumption of the developing solution is proportional to the concentration consumption of the acid in the photoresist. The consumption ratio of the acid in the photoresist and the developing solution can be determined according to the chemical reaction formula of the developing solution and the acid in the photoresist.

Step S32: creating the concentration distribution function D(x, y)=F(S(x, y)) according to the consumption ratio of the acid in the photoresist and the developing solution.

Additionally, the expression of the concentration distribution function of the developing solution is

$$\frac{\partial D}{\partial t} = - {K}_{amp} DS ,$$

where kₐₘₚ represents a crosslinking rate constant and t represents time.

As mentioned above, in the process of development, the developing solution will chemically react with the acid in the photoresist, and the developing solution will consume the acid in a corresponding proportion according to the chemical reaction formula. Therefore, it can be considered that the concentration consumption of the developing solution is proportional to the concentration consumption of the acid in the photoresist. Therefore, the concentration distribution function of the developing solution is created using the instantaneous consumption rate ∂D/∂t of the developing solution, which is highly correlated with the instantaneous consumption rate ∂G/∂t of the photoacid-producing agent. Then, the concentration distribution function D of the developing solution is correlated with the concentration distribution function S of the acid in the photoresist through the crosslinking reaction rate constant Kₐₘₚ.

Additionally, step S4 specifically comprises the following steps:

Step S41: creating an expression D1 representing a concentration diffusion direction of the developing solution.

Step S42: creating an expression D2 representing a concentration diffusion intensity of the developing solution.

It can be understood that, in the present disclosure, the developing solution in different positions has different concentrations, which may cause strong diffusion effect. The developing solution with a higher concentration may diffuse to the area where the developing solution with a lower concentration. When the concentration is greater than a certain intensity, the diffusion effect will be strengthened. The technical solution of the present disclosure can effectively and quickly calculate the sum of reactions by first creating the expression of the concentration diffusion direction of the developing solution and the expression of the concentration diffusion intensity of the developing solution, and then creating the calculation formula R of the concentration diffusion of the developing solution based on the idea of calculus, which greatly improves speed and accuracy of modeling.

Additionally, the expression representing a concentration diffusion direction of the developing solution is D1=D(x₁, y₁)*D(x₂, y₂), the expression representing a concentration diffusion intensity of the developing solution is D2=D(x₁, y₁)*(x₁−x₂, y₁−y₂). In the present disclosure, both the expression D1 representing a concentration diffusion direction of the developing solution and the expression D2 representing a concentration diffusion intensity of the developing solution are expressions related to (x, y). The variables are unified, which is convenient for operation, and can further reduce the amount of operation during modeling, and improve the operation speed.

Furthermore, step S4 specifically comprises the following steps:
obtaining the diffusion direction and the diffusion intensity of the developing solution in each unit area according to the expression D1 representing a concentration diffusion direction of the developing solution and the expression D2 representing a concentration diffusion intensity of the developing solution; and calculating diffusion results of the developing solution in each unit area based on the concentration distribution function D of the developing solution; constructing the calculation formula R(x, y) of the concentration diffusion of the developing solution based on the expression D1 representing a concentration diffusion direction of the developing solution and the expression D2 representing a concentration diffusion intensity of the developing solution; and adding up the diffusion results of the developing solution in each unit area to simulate diffusion of the developing solution.

It can be understood that the calculation formula R of the concentration diffusion of the developing solution concentration can accumulate the diffusion results of the developing solution in each unit area of the selected region, so as to obtain the total diffusion result of the developing solution. A high-accuracy negative tone development photoresist model can be obtained by optimizing existing computational negative tone development photoresist model according to the total diffusion result of the developing solution, which can take into account the modeling speed and accuracy, and can make the modeling speed comparable to the positive development while ensuring accuracy; In addition, the formula R for calculating concentration diffusion of the developing solution is based on the idea of calculus to accumulate the diffusion of concentration, and does not need to be calculated by pure brute force superposition, which further reduces the amount of calculation during modeling and improves the overall calculation speed.

Additionally, the formula for calculating the total diffusion result is R(x, y)=Σᵢ₌ₘ^{D(x}^{ᵢ}^{, y}^{ᵢ}^{)*D(x}^{₁}^{, y}^{₁}^{)<0}D(xᵢ, yᵢ)*(x₁−xᵢ, y₁−yᵢ), where m is a positive integer constant.

It can be understood that, the formula R(x, y)=Σᵢ₌ₘ^{D(x}^{ᵢ}^{, y}^{ᵢ}^{)*D(x}^{₁}^{, y}^{₁}^{)<0}D(xᵢ, yᵢ)*(x₁−xᵢ, y₁−yᵢ) is created based on the idea of calculus, which can quickly simulate the diffusion of the developing solution, so as to obtain the total diffusion result of the developing solution.

Additionally, in step S5, the change of photoresist in the development process is accurately simulated by accurately simulating the change of the concentration distribution of the developing solution in the development process, so as to obtain a high-precision negative tone development photoresist pattern after development.

Additionally, when the expression D1=D(x₁, y₁)*D((x₂, y₂) representing a concentration diffusion direction of the developing solution becomes a fixed value, that is, when there is no difference in the concentration, it indicates that the concentration distribution of the developing solution has entered a stable state and no longer diffuses.

Additionally, in step S6, if the comparison results of the relevant data between the negative tone development photoresist pattern and the preset pattern cannot meet the preset criteria, the high-precision photoresist pattern will be continued to be optimized until it reaches the preset criteria. It should be understood that, specific preset criteria can be set according to the type of data being compared.

Additionally, in the embodiment, the preset pattern in step S6 is a model design pattern; Comparation between the high-precision negative tone development photoresist pattern after development and relevant data of the preset pattern, including the deformation/error comparison of a key dimension, and the method for comparing the deformation/error of the key dimension includes comparing the root mean square of the key size; In addition, it can also compare the lattice error of key dimension. It can be understood that, the root mean square or the lattice error of the key dimension is as small as possible.

In other embodiments, other contrasting factors may also be added to the comparison.

It can be understood that in the present disclosure, the high-precision negative tone development photoresist pattern after development is compared with the preset pattern, and if the preset criteria cannot be satisfied, the modeling result will be continuously optimized, which is conducive to ensuring accuracy of the negative tone development photoresist model.

Specifically, in the embodiment, the preset criteria include whether the root mean square of the optimized model is less than 2; In other embodiments, the standard may also be other larger or smaller numerical value.

Furthermore, further optimization of the model includes further optimization of formulas such as S(x, y) and D(x, y).

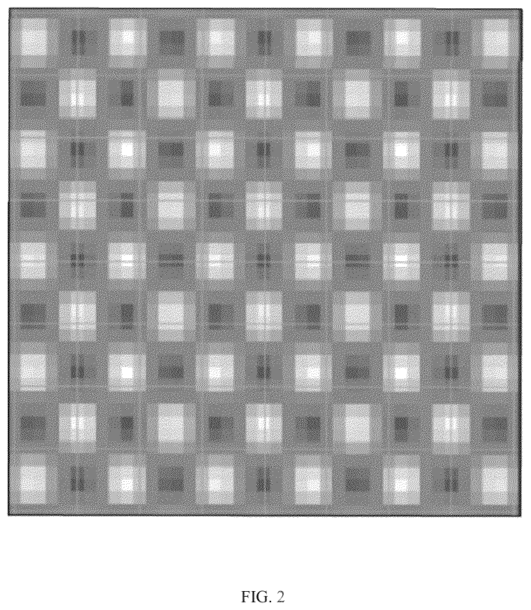

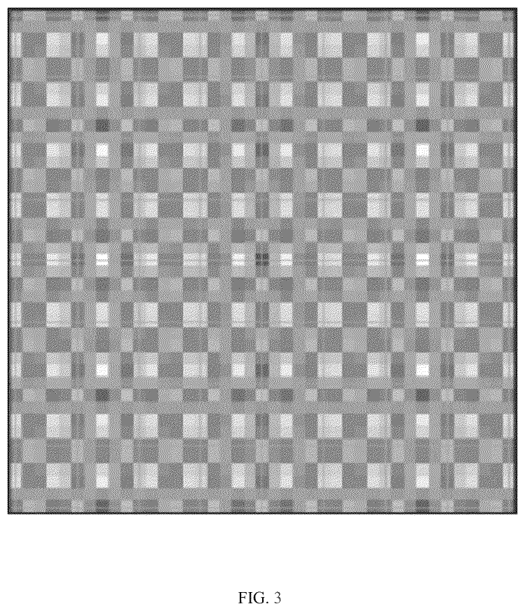

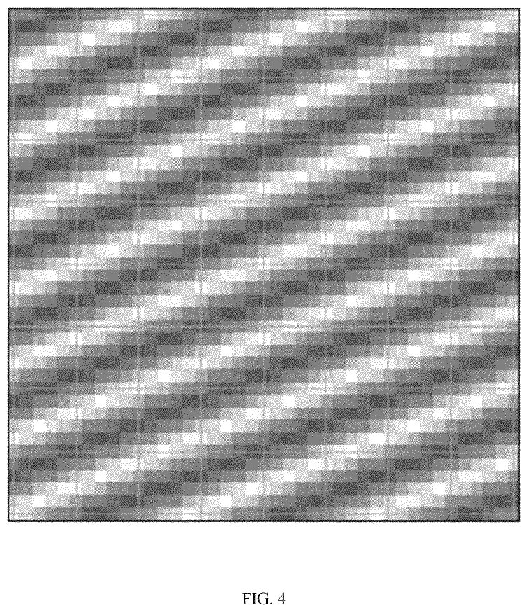

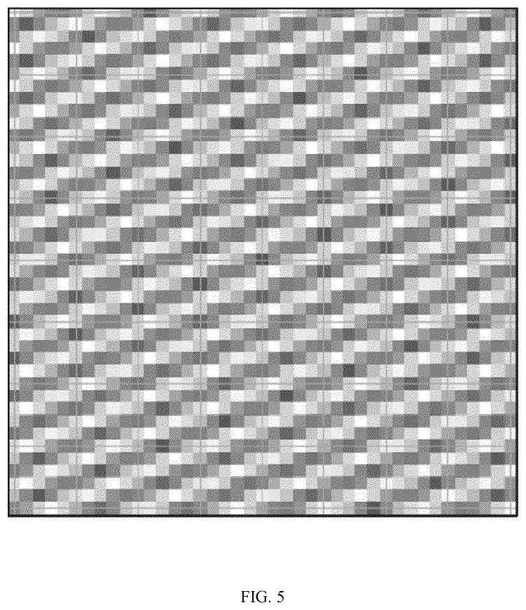

Please referring FIGS. 2-5, FIG. 2 represents the light field distribution diagram in the initially selected photoresist area, FIG. 3 represents the light field distribution diagram after the development effect on the photoresist due to different concentration of the developing solution, and different gray color blocks represent different light field intensities, through comparison of FIG. 2 and FIG. 3, it can be clearly seen that the image has changed very much after diffusion of the developing solution; Similarly, FIGS. 4 and 5 represent another set of comparison, including the light field distribution in the initially selected photoresist region (FIG. 4) and the light field distribution after the development effect on the photoresist due to different concentration of the developing solution (FIG. 5); And existing negative tone development photoresist model is unable to simulate the change from FIG. 2 to FIG. 3 or from FIG. 4 to FIG. 5, whereas the method for optimizing the negative tone development photoresist model of the present disclosure can optimize this, and the development process is simulated by the calculation formula R of the concentration diffusion of the developing solution, and the change of photoresist in the development process is accurately simulated by accurately simulating the change of the concentration distribution of the developing solution in the developing process, In this way, the high-precision negative tone development photoresist pattern after development is obtained.

Compared with the prior art, the method for optimizing the negative tone development photoresist model of the present disclosure has following advantages:
1. The method for optimizing the negative tone development photoresist model of the present disclosure is a method for simulating the negative tone developing based on concentration distribution of the developing solution. Firstly, the concentration distribution function S of the acid in the photoresist is created based on relevant data on the light field distribution in the initial negative tone development photoresist model; Then, in the process of development, the developing solution will chemically react with the acid in the photoresist, and the developing solution will consume the acid in a corresponding proportion according to the chemical reaction formula. Therefore, it can be considered that the concentration consumption of the developing solution is proportional to the concentration consumption of the acid in the photoresist, based on which, the concentration distribution function D of the developing solution can be quickly created; Concentration distribution data of the developing solution can be obtained based on the concentration distribution function D of the developing solution. The developing solution distributed in different regions have different concentrations, which will produce diffusion effects. Therefore, the formula R for calculating concentration diffusion of the developing solution can be created based on the concentration distribution function D of the developing solution. Since in the development process, the photoresist chemically reacts with the developing solution, the change of the concentration of the developing solution is directly related to the image of the photoresist. In the negative tone development process, the part of the photoresist reacting with the developing solution will be retained, therefore, it can be known that which regions of the photoresist undergoes the reaction and the image of the photoresist after the reaction is thus known. Therefore, we can accurately simulate the change of photoresist in the development process by accurately simulating the change of the concentration distribution of the developing solution during the development process, so as to obtain a pattern with a high-precision of the negative tone development photoresist after development. This method can take into account both modeling speed and accuracy, and can make the modeling speed of the negative tone development comparable to that of forward development while ensuring accuracy.
2. The light field distribution function E in the present disclosure is constructed according to the obtained data on the light field distribution, and the concentration distribution function S of the acid in the photoresist is constructed based on the light field distribution function E, so when the light field distribution function E is a function related to (x, y), the concentration distribution function S of the acid in the photoresist is also a function related to (x, y), and this design is convenient for unifying variables, making it convenient for operation, and is conducive to reducing the amount of operation during optimization, and to improve the operation speed; Moreover, the data on the light field distribution in the present disclosure is obtained on the basis of positions of the pixel points of the mask pattern, which has a high accuracy.
3. The acid in the photoresist in the present disclosure is produced by decomposition of the photoacid-producing agent, so the expression of the distribution function of the acid concentration in the photoresist is S=1−G. Since the photoacid-producing agent will be decomposed under the light to produce the acid, instantaneous consumption rate of the concentration of the photoacid-producing agent is relevant to the exposure rate constant c and the light field distribution function E.
4. The concentration distribution function D of the developing solution in the present disclosure is created according to the consumption ratio of the developing solution and the acid in the photoresist, and is a function related to (x, y), which is convenient for unifying variables, making it convenient for calculation, and is conducive to reducing the amount of operation during optimization, and improving the operation speed.
5. As mentioned in the first point above, in the process of development, the developing solution will chemically react with the acid in the photoresist, and the developing solution will consume the acid in a corresponding proportion according to the chemical reaction formula. Therefore, it can be considered that the concentration consumption of the developing solution is proportional to the concentration consumption of the acid in the photoresist. Therefore, the concentration distribution function of the developing solution is created using the instantaneous consumption rate ∂D/∂t of the developing solution, which is highly correlated with the instantaneous consumption rate ∂G/∂t of the photoacid-producing agent. Then, the concentration distribution function D of the developing solution is correlated with the concentration distribution function S of the acid in the photoresist through the crosslinking reaction rate constant Kₐₘₚ.
6. In the present disclosure, the developing solution in different positions has different concentrations, which may cause strong diffusion effect. The developing solution with a higher concentration may diffuse to the area where the developing solution with a lower concentration. When the concentration is greater than a certain intensity, the diffusion effect will be strengthened. The technical solution of the present disclosure can effectively and quickly calculate the sum of reactions by first creating the expression of the concentration diffusion direction of the developing solution and the expression of the concentration diffusion intensity of the developing solution, and then creating the calculation formula R of the concentration diffusion of the developing solution based on the idea of calculus, which greatly improves speed and accuracy of modeling.
7. In the present disclosure, both the expression D1 representing a concentration diffusion direction of the developing solution and the expression D2 representing a concentration diffusion intensity of the developing solution are expressions related to (x, y). The variables are unified, which is convenient for operation, and can further reduce the amount of operation during modeling, and improve the operation speed.
8. In the present disclosure, the formula R for calculating concentration diffusion of the developing solution is based on the idea of calculus to accumulate the diffusion of concentration, and does not need to be calculated by pure brute force superposition, which further reduces the amount of calculation during modeling and improves the overall calculation speed.
9. In the present disclosure, the developing state can be monitored by the expression D1=D(x₁, y₁)*D(x₂, y₂) of the concentration diffusion direction of the developing solution, and when the expression of the concentration diffusion direction of the developing solution becomes a fixed value, it indicates that the concentration distribution of the developing solution has entered a stable state and no longer diffuses.
10. The comparison factor in the present disclosure includes the root mean square and/or the grid point error of the preset key dimension, and the comparison is comprehensive, and the comparison error can be effectively avoided.

The method for optimizing negative tone development photoresist model disclosed in the embodiments of the present disclosure is described in detail. In this paper, specific examples are used to describe the principle and embodiments of the invention. The descriptions of the embodiments are only used to help understand the method and its core ideas of the invention. At the same time, for an ordinary person skilled in the art, according to the idea of the invention, there will be changes in a specific implementation mode and application ranges. The above description are only embodiments of the present disclosure, and is not intended to limit the present disclosure. As stated above, the content of the present disclosure cannot be considered to limit the present disclosure. Any modifications, equivalent substitutions, improvements, etc. made within the spirit and scope of the present disclosure are intended to be included within the scope of the present disclosure.

What is claimed is:

1. A method for optimizing a negative tone development photoresist model, comprising following steps:
obtaining an initial negative tone development photoresist model;
creating a concentration distribution function S of acid in the photoresist based on light field distribution in the initial negative tone development photoresist model and acid concentration in photoresist;
creating a concentration distribution function D of a developing solution based on the concentration distribution function S of the acid in the photoresist; wherein concentration consumption of the developing solution is proportional to concentration consumption of the acid in the photoresist;
creating a formula R for calculating concentration diffusion of the developing solution based on the concentration distribution function D of the developing solution to calculate diffusion results of developing solutions in different concentrations;
simulating a development process through the formula R for calculating concentration diffusion of the developing solution to obtain a pattern of simulated negative tone development photoresist after development; and
comparing relevant data of the pattern of simulated negative tone development photoresist with that of a preset pattern, and taking the pattern of simulated negative tone development photoresist as a formal pattern of negative tone development photoresist if preset criteria are met.

2. The method for optimizing a negative tone development photoresist model according to claim 1, wherein said creating the concentration distribution function S of the acid in the photoresist based on the light field distribution in the initial negative tone development photoresist model and the acid concentration in photoresist comprises following steps:
obtaining data on the light field distribution based on positions of pixel points of a mask pattern from the initial negative tone development photoresist model, and constructing a light field distribution function E(x, y) based on information on the positions of the pixel points according to obtained data on the light field distribution, where E is a function related to (x, y) and (x, y) represents a position of a pixel point; and
creating a concentration distribution function S(x, y) of the acid in the photoresist, the concentration distribution function S(x, y) of the acid in the photoresist is relevant to the light field distribution function E(x, y) and S(x, y)=F(E(x, y)).

3. The method for optimizing a negative tone development photoresist model according to claim 2, wherein the formula of the concentration distribution function S of the acid in the photoresist is S=1−G, where G is concentration of a photoacid-producing agent, an expression for instantaneous consumption rate of the concentration of the photoacid-producing agent is ∂G/∂t=−cGE, where c represents an exposure rate constant and t represents time.

4. The method for optimizing a negative tone development photoresist model according to claim 1, wherein said creating the concentration distribution function D of the developing solution based on the concentration distribution function S of the acid in the photoresist comprises following steps:
determining a consumption ratio of the acid in the photoresist and the developing solution according to a chemical reaction formula of the acid in the photoresist and the developing solution; and
creating the concentration distribution function D(x, y)=F(S(x, y)) according to the consumption ratio of the acid in the photoresist and the developing solution.

5. The method for optimizing a negative tone development photoresist model according to claim 4, wherein an expression of the concentration distribution function of the developing solution is
$$\frac{\partial D}{\partial t} = - {K}_{amp} DS ,$$
where Kₐₘₚ represents a crosslinking rate constant and t represents time.

6. The method for optimizing a negative tone development photoresist model according to claim 1, wherein, said creating the formula R for calculating concentration diffusion of the developing solution based on the concentration distribution function D of the developing solution to calculate diffusion results of developing solutions in different concentrations comprises following steps:
creating an expression D1 representing a concentration diffusion direction of the developing solution; and
creating an expression D2 representing a concentration diffusion intensity of the developing solution.

7. The method for optimizing a negative tone development photoresist model according to claim 6, wherein the expression representing a concentration diffusion direction of the developing solution is D1=D(x₁, y₁)*D(x₂, y₂), the expression representing a concentration diffusion intensity of the developing solution is D2=D(x₁, y₁)*(x₁−x₂, y₁−y₂).

8. The method for optimizing a negative tone development photoresist model according to claim 7, wherein said creating the formula R for calculating concentration diffusion of the developing solution based on the concentration distribution function D of the developing solution to calculate diffusion results of developing solutions in different concentrations comprises following steps:
obtaining the diffusion direction and the diffusion intensity of the developing solution in each unit area according to the expression D1 representing a concentration diffusion direction of the developing solution and the expression D2 representing a concentration diffusion intensity of the developing solution; calculating diffusion results of the developing solution in each unit area based on the concentration distribution function D of the developing solution; constructing the calculation formula R(x, y) of the concentration diffusion of the developing solution based on the expression D1 representing a concentration diffusion direction of the developing solution and the expression D2 representing a concentration diffusion intensity of the developing solution; and
adding up the diffusion results of the developing solution in each unit area to simulate diffusion of the developing solution.

9. The method for creating a negative tone development photoresist model according to claim 8, wherein the formula for calculating a total diffusion result is R(x, y)=Σᵢ₌ₘ^{D(x}^{ᵢ}^{, y}^{ᵢ}^{)*D(x}^{₁}^{, y}^{₁}^{)<0}D(xᵢ, yᵢ)*(x₁−xᵢ, y₁−yᵢ), where m is a positive integer constant; and the formula R(x, y)=Σᵢ₌ₘ^{D(x}^{ᵢ}^{, y}^{ᵢ}^{)*D(x}^{₁}^{, y}^{₁}^{)<0}D(xᵢ, yᵢ)*(x₁−xᵢ, y₁−yᵢ) is created based on an idea of calculus.

10. The method for creating a negative tone development photoresist model according to claim 7, wherein comparison factors for comparing the pattern of simulated negative tone development photoresist with that of the preset pattern comprises root mean square and/or grid point error of a preset key dimension.

11. The method for creating a negative tone development photoresist model according to claim 10, wherein the preset criteria comprise root mean square of an optimized model is less than 2.

12. The method for creating a negative tone development photoresist model according to claim 5, wherein the concentration distribution function of the developing solution is created using the instantaneous consumption rate of the developing solution.

13. The method for creating a negative tone development photoresist model according to claim 12, wherein the concentration distribution function D of the developing solution is correlated with the concentration distribution function S of the acid in the photoresist through the crosslinking reaction rate constant Kₐₘₚ.
